(19) United States
(12) Patent Application Publication
(10) Pub. No.: US 2021/0052566 A1
(43) Pub. Date: Feb. 25, 2021

(54) METHODS FOR TREATING HEPATITIS B VIRUS (HBV) INFECTION

(71) Applicants: INSERM (INSTITUT NATIONAL DE LA SANTÉ ET DE LA RECHERCHE MÉDICALE), Paris (FR); UNIVERSITÉ CLAUDE BERNARD - LYON 1, Villeurbanne (FR); CENTRE NATIONAL DE LA RECHERCHE SCIENTIFIQUE (CNRS), Paris (FR); CENTRE LEON BERARD, Lyon (FR); UNIVERSITY OF BRITISH COLUMBIA, Vancouver (CA); SOCPRA SCIENCES SANTE ET HUMAINE S E C, Sherbrooke (CA)

(72) Inventors: Anna SALVETTI, Lyon Cedex 08 (FR); David DURANTEL, Lyon Cedex 08 (FR); Hélène CHABROLLES, Lyon Cedex 08 (FR); Tomas LAHLALI, Lyon Cedex 08 (FR); David GRIERSON, Vancouver (CA); Benoit CHABOT, Sherbrook (CA)

(21) Appl. No.: 17/043,978

(22) PCT Filed: Sep. 27, 2018
(86) PCT No.: PCT/EP2018/076346

§ 371 Date: Sep. 30, 2020

(30) Foreign Application Priority Data
Apr. 4, 2018 (EP) 18305398.2

(51) Int. Cl. A61K 31/4439; A61P 31/20

(52) U.S. Cl. CPC A61K 31/4439; A61P 31/20

(57) ABSTRACT

In the present invention, the proteomic identification of HBc interacting factors in the nucleus of human hepatocytes revealed a majority of RNA-binding proteins (RBPs) intervening in mRNA metabolism and especially, the serine/arginine-rich splicing factor (10) (SRSF1O) which was found enriched nearly (3000) times in HBc complexes. Inventors demonstrated that the inhibition of SRSF1O phosphorylation with the small molecule 1C8 (4-pyridinonebenzisothiazole carboxamide) induces a strong inhibition of HBV replication (genotypes C and D) in persistently-infected hepatocytes, as well as to a strong inhibition of the establishment of HBV cccDNA in de novo infection settings. Accordingly the present invention relates to an inhibitor of SRSF1O activity for use in the treatment of Hepatitis B virus (HBV) infection said inhibitor maintain SRSF1O in a dephosphorylated state and prevents or reduces the splicing activity of SRSF1O.

### FIELD OF THE INVENTION

The present invention relates methods and pharmaceutical compositions for treating Hepatitis B infection, using an inhibitor of biological activity of SRSF10 (serine/arginine-rich splicing factor 10) in particular said inhibitor maintains SRSF10 in a dephosphorylated state, and prevents or reduces the splicing activity of SRSF10.

### BACKGROUND OF THE INVENTION

Despite the existence of a preventive vaccine, chronic Hepatitis B virus (HBV) infections remain a major health problem worldwide, as they concern 250 millions individuals and represent the first cause of primary liver cancer (hepatocellular carcinoma, HCC) (Petruzziello, 2018). Current clinically accepted antiviral treatments against HBV chronic infections consist of pegylated interferon-α (IFN-α), which directly targets transcription from viral DNA and indirectly boosts host immune responses, and/or nucleoside analogs (NUC), which specifically inhibit HBV reverse transcription. These treatments generally lead to a transient (for IFN) or long-lasting (for NUC) reduction of viremia in the blood of patients (Zoulim and Durantel, 2015). However, the liver viral clearance is rarely obtained, thus making life-long therapy with NUC mandatory. There is therefore an urgent need to develop new classes of antiviral molecules able to inhibit HBV replication either directly or indirectly, which could be used in combinatorial therapies.

In cells highly replicating HBV, the HBV core protein (HBc) accumulates in the nucleus, meaning that besides its well-known role in the encapsidation of pgRNA and subsequent reverse transcription to generate virion genomes, HBc could have other regulatory functions. Early studies have shown that HBc binds to HBV cccDNA as well as to cellular DNA (Zlotnick et al., 2015). These findings suggested that this viral protein may regulate the expression of the viral mini-chromosome (cccDNA) by recruiting epigenetic modulators and/or preventing the access to silencing factors. However, more recent studies suggest that newly synthetized HBc is not required for HBV transcription itself. Therefore, HBc nuclear functions still remain undefined (Seeger et al. 2015).

To decipher HBc nuclear functions inventors identified its nuclear partners in human differentiated hepatocytes. The proteomic identification of HBc interacting factors in the nucleus of human hepatocytes revealed a majority of RNA-binding proteins (RBPs) intervening in mRNA metabolism. Notably, the serine/arginine-rich splicing factor 10 (SRSF10) was found enriched nearly 3000 times in HBc complexes thus highlighting a strong and preferential interaction of HBc with this nuclear RNA-binding factor.

In order to further investigate the role of SRSF10 during HBV replication inventors used the compound 1C8 (4-pyridinonebenzisothiazole carboxamide), which was recently identified as a strong inhibitor of HIV-1 replication (Shkreta et al., 2017). 1C8 was initially identified as structural mimic of IDC16, a drug inhibiting the splicing function of SRSF1, a major SR factor (Cheung et al., 2016). Molecular analyses indicated that 1C8 does not inhibit SRSF1 but rather prevents the phosphorylation of SRSF10 and thus modulates its splicing activities (Shkreta et al., 2017). In particular, 1C8 was shown to inhibit SRSF10 phosphorylation at serine 133. Phosphorylation of this residue, together with that occurring at serine 131 was previously found to alter the interaction with other splicing factors (Shkreta et al., 2017). In vitro studies indicated that 1C8 strongly inhibits HIV replication with little impact on cellular genes and cell viability.

Inhibition of HIV replication was correlated to the alteration in the synthesis of spliced viral mRNA and to a decrease of SRSF10 binding to HIV mRNAs (Shkreta et al., 2017). The patent application WO 2015/164956 discloses benzisothiazole derivative compounds (to which belongs 1C8) and their use for the treatment of HIV infections.

### SUMMARY OF THE INVENTION

In the present invention, inventors studying the mechanism of HBV replication tried to decipher HBc regulatory functions, by identifying its cellular partners in the nucleus of human hepatocytes by a mass spectrometry (MS) analysis. This analysis identified more than 200 proteins. Functional annotation revealed that approximately 50% of these factors were RNA binding proteins (RBPs), and in particular SR proteins, with the most relevant functional category corresponding to factors involved in RNA splicing which were highly interconnected. Functional analyses identified SRSF10 as able to strongly and differentially modulate HBV replication. In addition, the small compound 1C8 targeting the activity of SRSF10 proteins induced a strong decrease of viral replication with different strains of HBV (genotypes C and D).

Accordingly the present invention relates to an inhibitor of SRSF10 activity for use in the treatment of Hepatitis B virus (HBV) infection.

In particular embodiment, said inhibitor maintains SRSF10 in a dephosphorylated state, and prevents or reduces the splicing activity of SFR10.

In particular embodiment, the inhibitor of SRSF10 activity is capable of reducing cccDNA and/or pgRNA in an infected cell.

The present invention also relates to a method for screening a plurality of candidate compounds useful for treating Hepatitis B virus (HBV) infection comprising the steps consisting of (a) testing each of the candidate compounds for its ability to inhibit SRSF10 activity and (b) and positively selecting the candidate compounds capable of inhibiting said SRSF10 activity.

### DETAILED DESCRIPTION OF THE INVENTION

In the present study the inventors demonstrate that SRSF10 is a main factor present in HBc nuclear complexes and could therefore play a role in the HBV life cycle. As 1C8 was shown to modulate the phosphorylation status of SRSF10, inventors have straightforwardly investigated whether this molecule, which was already shown to modulate the replication of HIV, could also inhibit the replication of HBV. They found that 1C8 compound is indeed capable to inhibit the replication of HBV (genotypes D and C) in persistently-infected hepatocytes by strongly reducing intracellular HBV RNAs by 60-70% (see FIG. 5A), as well as the secretion of viral antigens (HBs and HBeAg) and HBV virions (see FIG. 5B) in cell culture supernatant by 60-70%. IC8 compound was also, capable to inhibit the establishment of HBV cccDNA in de novo infection setting: addition of the molecule on hepatocytes before and during HBV inoculation (genotypes C and D) strongly reduces cccDNA establishment (respectively by 70 and 50%) and other viral markers (i.e. intracellular RNAs, HBeAg, HBsAg) were also reduced in 1C8-treated conditions (FIGS. 7A and B and FIGS. 8A and B).

HBV cccDNA in infected hepatocytes is responsible for persistent chronic infection and reactivation, being the template for all viral subgenomic transcripts and pre-genomic RNA (pgRNA) to ensure both newly synthesized viral progeny and cccDNA pool replenishment via intracellular nucleocapsid recycling. In the context of the present invention it was for the first time shown that SRSF10 activity controls 1) the fate of intracellular HBV RNA (HBV total RNA and HBV pregenomic RNA) in chronically infected cells and 2) cccDNA establishment in de novo infection. This knowledge provides the opportunity to reduce de novo cccDNA synthesis in HBV infected subjects, which in turn opens the opportunity for a complete cure of chronically infected HBV patients.

Altogether, these results indicate that the inhibition of SRSF10 phosphorylation induces a strong inhibition of HBV infection in hepatic cells and represents an attractive new therapy for HBV infections.

Accordingly, the present invention relates to an inhibitor of SRSF10 activity for use in the treatment of Hepatitis B virus (HBV) infection.

In particular embodiment, said inhibitor maintains SRSF10 in a dephosphorylated state, and prevents or reduces the splicing activity of SFR10.

In particular embodiment, the inhibitor of SRSF10 activity is capable of reducing cccDNA and/or pgRNA in an infected cell.

As used herein, the term “HBV infection” refers to an infectious disease commonly known in the art that is caused by the hepatitis B virus (HBV) and affects the liver. HBV infection can be an acute or a chronic infection. Some infected persons have no symptoms during the initial infection and some develop a rapid onset of sickness with vomiting, yellowish skin, tiredness, dark urine and abdominal pain (“Hepatitis B Fact sheet No 204”. who.int. July 2014. Retrieved 4 Nov. 2014). Often these symptoms last a few weeks and can result in death. It may take 30 to 180 days for symptoms to begin. In those who get infected around the time of birth 90% develop a chronic hepatitis B infection while less than 10% of those infected after the age of five do (“Hepatitis B FAQs for the Public—Transmission”, U.S. Centers for Disease Control and Prevention (CDC), retrieved 2011-11-29). Most of those with chronic disease have no symptoms; however, cirrhosis and liver cancer may eventually develop (Chang, 2007, Semin Fetal Neonatal Med, 12: 160-167). These complications result in the death of 15 to 25% of those with chronic disease (“Hepatitis B Fact sheet No 204”. who.int. July 2014, retrieved 4 Nov. 2014). Herein, the term “HBV infection” includes the acute and chronic hepatitis B infection. The term “HBV infection” also includes the asymptotic stage of the initial infection, the symptomatic stages, as well as the asymptotic chronic stage of the HBV infection.

In particular embodiment HBV infection is a chronic infection.

The hepatitis B virus (HBV) is an enveloped, partially double-stranded DNA virus. The compact 3.2 kb HBV genome consists of four overlapping open reading frames (ORF), which encode for the core, polymerase (Pol), envelope and X-proteins. The Pol ORF is the longest and the envelope ORF is located within it, while the X and core ORFs overlap with the Pol ORF. The lifecycle of HBV has two main events: 1) generation of closed circular DNA (cccDNA) from relaxed circular (RC DNA), and 2) reverse transcription of pregenomic RNA (pgRNA) to produce RC DNA.

As used herein, the term cccDNA (covalently closed circular DNA) is a special DNA structure that arises during the propagation of some DNA viruses in the cell nucleus. The cccDNA is also known as episomal DNA or occasionally as a minichromosome. cccDNA is typical of Hepadnaviridae, including the hepatitis B virus (HBV). The HBV genome forms a stable minichromosome, the covalently closed circular DNA (cccDNA), in the hepatocyte nucleus. The cccDNA is formed by conversion of capsid-associated relaxed circular DNA (rcDNA). HBV cccDNA formation involves a multi-step process that requires the cellular DNA repair machinery and relies on specific interactions with distinct cellular components that contribute to the completion of the positive strand DNA in rcDNA (Alweiss et al. 2017, Viruses, 9 (6): 156). HBV genome forms a stable minichromosome, the covalently closed circular DNA (cccDNA), in the hepatocyte nucleus. The cccDNA is formed by conversion of capsid-associated relaxed circular DNA (rcDNA). HBV cccDNA formation involves a multi-step process that requires the cellular DNA repair machinery and relies on specific interactions with distinct cellular components that contribute to the completion of the positive strand DNA in rcDNA (Alweiss et al. 2017, Viruses, 9 (6): 156).

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a subject, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

Accordingly, herein “treating a HBV infection” includes treating and preventing a HBV infection from occurring in a subject, and treating and preventing the occurrence of symptoms of a HBV infection. In the present invention in particular the prevention of HBV infection in children from HBV infected mothers are contemplated. Also contemplated is the prevention of an acute HBV infection turning into a chronic HBV infection.

The terms “subject,” and “patient,” used interchangeably herein, refer to a mammal, particularly a human who has been previously diagnosed with Fibrotic interstitial lung diseases such as idiopathic pulmonary fibrosis or who is at risk for having or developing idiopathic pulmonary fibrosis. Typically, a diagnosis of idiopathic pulmonary fibrosis may be made after lung biopsy or by using high resolution computed tomography (HRCT).

As used herein the term “serine/arginine-rich splicing factor 10” or “SRSF10” has its general meaning in the art. SRSF10 (also known as NSSR; TASR; SRp38; TASR1; TASR2; FUSIP1; FUSIP2; SFRS13; SRrp40; SFRS13A; PPP1R149) belongs to the family of SR (serine/arginine) proteins, a group of RBPs (RNA-binding proteins) involved in premRNA splicing. It is encoded in human by a gene (ID: 10772) located on chromosome 1 at position 1p36.11. SRSF10 was initially identified as a general splicing repressor upon its dephosphorylation during mitosis and in response to a heat shock (Shin et al., 2004; Shin et al., 2005). In contrast, when phosphorylated SRSF10 can activate splicing and its phosphorylation status determines its interaction with diverse RBPs such as hnRNPs (hnRNPK, F, and H) and TRA2B (Shkreta et al., 2017; Shkreta et al. 2016). Importantly, when phosphorylated SRSF10 activates alternative splicing of several cellular transcripts linked to pathways of stress, DNA damage, apoptosis, and carcinogenesis (Shkreta et al., 2016; Zhou et al., 2014a; Zhou et al., 2014b). Finally, SRSF10 also plays a role in the splicing of viral premRNAs, in particular HIV-1 transcripts (Shkreta et al., 2017).

Inhibitor of SRSF10 Activity

An “inhibitor of SRSF10 activity” has its general meaning in the art, and refers to a compound (natural or not), which has the capability of reducing or suppressing the biological activity of SRSF10. In the present application, said compound maintain SRSF10 in a dephosphorylated state, and prevents or reduces the splicing activity of SFR10. For example the compound may maintain or block SRSF10 in a dephosphorylated state in manner that SRSF10 is not able to activate splicing of viral and cellular RNAs and to bind with diverse RBPs (see Shkreta et al., 2017; Shkreta et al., 2016), which results to reduce cccDNA and/or pgRNA in an infected cell. Typically, said inhibitor is a small organic molecule or a biological molecule (e.g. peptides, lipid, aptamer, antibody . . . ).

By “biological activity” of phosphorylated SRSF10 is meant inducing its splicing activity on HBV infected cells associated with the establishment of cccDNA and/or pgRNA in an infected cell.

Tests for determining the capacity of a compound to be inhibitor of SRSF10 activity are well known to the person skilled in the art. In a preferred embodiment, the inhibitor specifically maintains SRSF10 in a dephosphorylated state in a sufficient manner to inhibit the biological activity of SRSF10. Maintaining the SRSF10 in a dephosphorylated state and inhibition of the biological activity of SRSF10 may be determined by any assays well known in the art. For example the assay may consist in determining the ability of the agent to alter the phosphorylate status of SRSF10. A classical method of directly measuring protein phosphorylation involves the incubation of whole cells with radiolabeled 32P-orthophosphate, the generation of cellular extracts, separation of proteins by SDS-PAGE, and exposure to film. Other traditional methods include 2-dimensional gel electrophoresis, a technique that assumes phosphorylation will alter the mobility and isoelectric point of the protein. For instance in Shkreta et al 2016 and Shkreta et al 2017, an assay for phosphorylation status of SRSF10 is described based on faster mobility in gel conditions of dephosphorylated version of SRSF10 compared with phosphorylated forms. Liquid chromatography-mass spectrometry (LC-MS) are also useful tools for assessing the phosphorylate status of SRSF10. Shkreta et al 2017 also use this assay to determine that IC8 promote the dephosphorylation of SRSF10 at serine 133.

Then a competitive assay may be settled to determine the ability of the agent to inhibit biological activity of SRSF10. The functional assays may be envisaged such evaluating the ability to induce or inhibit the splicing activity in HBV infected cells associated with the establishment of cccDNA and/or pgRNA in an infected cell (see example with 1C8 compound and FIGS. 7-8).

The skilled in the art can easily determine whether an SRSF10 activity inhibitor neutralizes, blocks, inhibits, abrogates, reduces or interferes with a biological activity of the SRSF10. To check whether the SRSF10 activity inhibitor alters the phosphorylation status of SRSF10 and/or inhibits splicing activity of SFR10 and/or inhibit cccDNA and/or pgRNA in HBV infected cell in the same way than the initially characterized 1C8 compound may be performed with each inhibitor. For instance HBV infection in hepatic cells can be measured by analysis of viral parameters such as cccDNA quantification and/or pgRNA quantification. For cccDNA quantification total DNA is digested by T5 exonuclease (New England Biolabs) then submitted to qPCR using Taqman Fast Advanced Master Mix (Life Technologies. For pgRNA quantification, RT-qPCR may be performed using Taqman Fast Advanced Master Mix (Life Technologies). PgRNA levels is normalized to GUSB using a commercial probe primer mix (Life Technologies #Hs99999908_m1).

HBV infection can also be measured by analysis of viral parameters such as quantification of secreted HBe and HBs antigens by Elisa (chemiluminescence immunoassay kit Autobio,): level of secreted HBe and HBs antigens are standard secreted markers of HBV infection of hepatic cells) and/or assessment of Intracellular total HBV DNA or RNA extracted from infected cells by qPCR or RT-qPCR with specific HBV primers as described in example section

Small molecules inhibiting SRSF10 have been identified in relation to SRSF10's role in HIV; such SRSF10 inhibitors are also envisioned as useful in the present invention of treating HBV. In particular targeting of such small molecule compounds, e.g. via conjugation or formulation, to the liver may be beneficial in the treatment of HBV

In a particular embodiment, the activity inhibitor according to the invention is a small organic molecule such as compound 1C8 (or C8): 4-pyridinonebenzisothiazole carboxamide (see Shkreta et al 2016 and Shkreta et al 2017 WO2015164956) or the benzisothiazole derivative compounds (see WO2015164956) to which belongs 1C8. All the active compounds disclosed in WO2015164956 are hereby incorporated by reference. In particular the following compounds

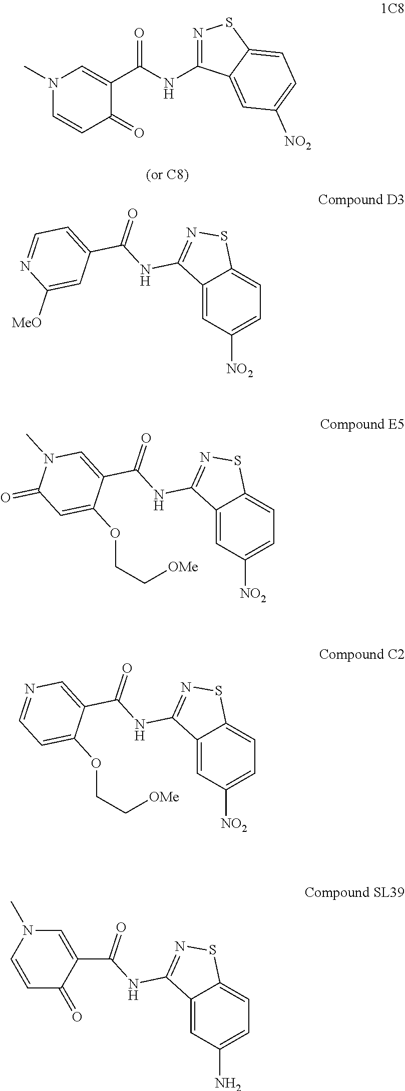 [SMILES: Cn1ccc(=O)c(C(=O)Nc2nsc3ccc([N+](=O)[O-])cc23)c1 ; COc1cc(C(=O)Nc2nsc3ccc([N+](=O)[O-])cc23)ccn1 ; COCCOc1cc(=O)n(C)cc1C(=O)Nc1nsc2ccc([N+](=O)[O-])cc12 ; COCCOc1ccncc1C(=O)Nc1nsc2ccc([N+](=O)[O-])cc12 ; Cn1ccc(=O)c(C(=O)Nc2nsc3ccc(N)cc23)c1]

All these compounds show an anti HIV activity especially the most active the 1C8 compound which inhibits the phosphorylation of SRSF10 at residue 133 and thus modulates its splicing activities.

In one embodiment, the present invention also relates to benzisothiazole derivative compounds for use in the treatment of Hepatitis B virus (HBV) infection.

The benzisothiazole derivative compounds are disclosed in WO2015164956 and are hereby incorporated by reference. In a preferred embodiment said compound is selected from the list consisting of: 1C8, E5, D3, C2 or SL309 compound.

In a particular embodiment, the present invention also relates to compound 1C8 (4-pyridinonebenzisothiazole carboxamide) or the benzisothiazole derivative compounds (E5, D3, C2 or SL309 compounds) for use in the treatment of Hepatitis B virus (HBV) infection.

Pharmaceutical Compositions

The inhibitor of SRSF10 activity may be combined with pharmaceutically acceptable excipients, and optionally sustained-release matrices, such as biodegradable polymers, to form therapeutic compositions.

In the pharmaceutical compositions of the present invention for oral, sublingual, subcutaneous, intramuscular, intravenous, transdermal, local, inhaled or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The inhibitor of SRSF10 activity of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compound in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media, which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The inhibitor of SRSF10 activity or expression of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; timerelease capsules; and any other form currently used.

Method of Screening

The present invention also relates to a method for screening a plurality of candidate compounds useful for treating Hepatitis B virus (HBV) infection comprising the steps consisting of (a) testing each of the candidate compounds for its ability to inhibit SRSF10 activity and (b) and positively selecting the candidate compounds capable of inhibiting said SRSF10 activity.

Typically, the candidate compound is selected from the group consisting of small organic molecules, peptides, polypeptides or oligonucleotides.

Testing whether a candidate compound can inhibit SRSF10 activity can be determined using or routinely modifying reporter assays known in the art.

For example, the method may involve contacting cells expressing SRSF10 with the candidate compound, and measuring the phosphorylation status of SRSF10 (e.g., activation or repression of SRSF10 splicing activities), and comparing the cellular response to a standard cellular response. Typically, the standard cellular response is measured in absence of the candidate compound. A decrease cellular response over the standard indicates that the candidate compound is an inhibitor of SRSF10 activity.

The candidate compounds that have been positively selected may be subjected to further selection steps in view of further assaying its properties on hepatocytes cells isolated from subjects suffering from HBV infections (or dHeparRG cells infected by HBV see Grippon et al 2002). For example, the candidate compounds that have been positively selected with the screening method as above described may be further selected for their ability to inhibit the splicing activity of SRSF10 in HBV infected cells or reducing cccDNA and/or pgRNA in HBV infected cell. Typically, the screening method may further comprise the steps of i) bringing into contact hepatocytes from patients with HBV infection with a positively selected candidate compound ii) determining the amount of cccDNA and/or pgRNA in said HBV infected cell and iii) comparing the amount of cccDNA and/or pgRNA determined at step ii) with the amount of cccDNA and/or pgRNA determined when step i) is performed in the absence of the positively selected candidate compound. Step i) as above described may be performed by adding an amount of the candidate compound to be tested to the culture medium of the hepatocytes cells. Usually, a plurality of culture samples are prepared, so as to add increasing amounts of the candidate compound to be tested in distinct culture samples. Generally, at least one culture sample without candidate compound is also prepared as a negative control for further comparison.

Finally, the candidate compounds that have been positively selected may be subjected to further selection steps in view of further assaying its properties on animal models for HBV infections. Typically, the positively selected candidate compound may be administered to the animal model and the progression of HBV infections is determined and compared with the progression of HBV infections is in an animal model that was not administered with the candidate compound.

The invention will be further illustrated by the following figures and examples. However, these examples and figures should not be interpreted in any way as limiting the scope of the present invention.

### FIGURES

FIG. 1. Experimental outlines. Treatment schemes: (a) Treatments in pre-infection: dHepaRG cells were treated for 24 h (at day −1) with 100 nM of PreS1 peptide (PreS1-pep) or 10 μM of 1C8, or untreated (NT). At day 0, cells were inoculated with HBV (MOI=250) or with HDV (MOI=25) in the presence of drugs for 24 h. For the tenofovir control (TDF at 10 μM), cells were treated at day 1 and 4 post-infection. Supernatant and cells were harvested at day 7 post-infection. (b) Treatments in post-infection: dHepaRG cells were inoculated with HBV (MOI=250) or with HDV (MOI=25). At days 4, 7 and 9 post infection, cells were treated with TDF 10 μM, lamivudine (3TC) at 10 μM, 1C8 10 μM or untreated (NT). Supernatant and cells were harvested at day 11 postinfection.

FIG. 2. Schematic of the purification procedure of StepTag-HBc complexes. HepaRG-TR-StrepTag-HBc cells were grown and differentiated in large quantity (100 millions cells). After tetracycline induction, nuclei of cells were recovered and lysed with appropriate buffer (c.f. to IBA protocol). The nuclear fractions were either treated with benzonase (i.e., digestion of DNA and RNA to avoid nucleic acid bridging) or left untreated, then passed onto the StrepTactin column. The elution fraction 2 was sent-out for LC-MS/MS analysis

FIG. 3. Result of the interactome study and primary validation

FIG. 4. Enrichment scores of ST-HBc associated proteins

FIG. 5. Anti-HBV properties of 1C8 in persistently infected dHepaRG cells: genotype D. Differentiated HepaRG were infected with HBV genotype D at a MOI of 100 vge/cell or HDV genotype 1 at a MOI of 10 vge/cell for 7 days before being treated trice every 2-3 days with indicated molecules at a concentration of 10 microM. A) Total DNA and RNA were extracted from cells and subjected to qPCR and RTqPCR to detect HBV cccDNA, total HBV RNA or pregenomic HBV RNA. B) HBs and HBe secreted antigens in supernatant were quantified by ELISA, whereas HBV DNA contained in virions was quantified by qPCR after viral nucleic acid extraction from supernatant. C) Intracellular HDV RNA was quantified by RTqPCR after total RNA extraction from cells. D) Viability was monitored in 1C8-treated (or control treated; puromycin is a positive toxic molecule) cells by CellTiter-Glo® luminescent cell viability assay. Results are the mean+SEM of n=3 performed with 3 different differentiations of HepaRG. Statistics were done by two-tailed Mann-Whitney test, with following significativity: *P<0.05, **P<0.01, ***P<0.001, ****P<0.0001. nd=not determined

FIG. 6. Anti-HBV properties of 1C8 in persistently infected dHepaRG cells: genotype C. Differentiated HepaRG were infected with HBV genotype C at a MOI of 100 vge/cell for 7 days before being treated trice every 2-3 days with indicated molecules at a concentration of 10 microM. A) Total DNA and RNA were extracted from cells and subjected to qPCR and RTqPCR to detect HBV cccDNA, total HBV RNA or pregenomic HBV RNA. B) HBs and HBe secreted antigens in supernatant were quantified by ELISA, whereas HBV DNA contained in virions was quantified by qPCR after viral nucleic acid extraction from supernatant. Results are the mean+SEM of n=3 performed with 3 different differentiations of HepaRG. Statistics were done by two-tailed Mann-Whitney test, with following significativity: *P<0.05, **P<0.01, ***P<0.001, ****P<0.0001. nd=not determined.

FIG. 7. Effect of 1C8 on de novo establishment of ccDNA in dHepaRG (genotype D). Differentiated HepaRG were treated 24 hours prior inoculation and during the 24 hours of inoculation with indicated drugs at 10 microM (C8 and TDF) or at 100 nM (Entry Inh.), then were infected with HBV genotype D at a MOI of 100 vge/cell or HDV genotype 1 at a MOI of 10 vge/cell for 7 days. A) Total DNA and RNA were extracted from cells and subjected to qPCR and RTqPCR to detect HBV cccDNA, total HBV RNA or pregenomic HBV RNA. B) HBs and HBe secreted antigens in supernatant were quantiUied by ELISA, whereas HBV DNA contained in virions was quantified by qPCR after viral nucleic acid extraction from supernatant. C) Intracellular HDV RNA was quantiUied by RTqPCR after total RNA extraction from cells. Results are the mean+SEM of n=3 performed with 3 different differentiations of HepaRG. Statistics were done by two-tailed Mann-Whitney test, with following signiUicativity: *P<0.05, **P<0.01, ***P<0.001, ****P<0.0001. nd=not determined.

FIG. 8. Effect of 1C8 on de novo establishment of cccDNA in dHepaRG (genotype C). Differentiated HepaRG were treated 24 hours prior inoculation and during the 24 hours of inoculation with indicated drugs at 10 microM (1C8 and TDF) or at 100 nM (Entry Inh.), then were infected with HBV genotype C at a MOI of 100 vge/cell for 7 days. A) Total DNA and RNA were extracted from cells and subjected to qPCR and RTqPCR to detect HBV cccDNA, total HBV RNA or pregenomic HBV RNA. B) HBs and HBe secreted antigens in supernatant were quantified by ELISA, whereas HBV DNA contained in virions was quantified by qPCR after viral nucleic acid extraction from supernatant. Results are the mean+SEM of n=3 performed with 3 different differentiations of HepaRG. Statistics were done by two-tailed Mann-Whitney test, with following significativity: *P<0.05, **P<0.01, ***P<0.001, ****P<0.0001. nd=not determined

FIG. 9. Effect of 1C8 on persistently infected primary human hepatocytes. Two different batches of PHH from two different donors were used in these experiments (Batch 1: panels A, B, and C; Batch 2: panels D, and E). A) PHH were infected with HBV genotype D at a MOI of 100 vge/cell for 4 days before being treated trice every 2-3 days with indicated molecules at a concentration of 10 microM. B) Intracellular total RNA were extracted from cells and subjected to RT-qPCR to detect HBV pgRNA and total HBV RNA. C) HBs and HBe secreted antigens in supernatant were quantified by ELISA. D) PHH were infected with HBV genotype D at a MOI of 100 vge/cell. 6 days after the infection, cells were treated five times with indicated molecules at a concentration of 10 microM. Supernatant was harvested after each treatment and cells were harvested at day 13 post infection. E) HBe and HBs secreted antigens in supernatant were quantified by ELISA. Results are the mean+SD of n=3 (panels A, B, and C) or n=4 (panels D and E).

### EXAMPLE 1

Material & Methods:

Cultivation of Wild-Type HepaRG, Engineered HepaRG Cells, and Condition of HBV Infection.

Human liver progenitor HepaRG cells were cultured in Williams medium (ThermoFisher) supplemented with 10% of FBS (Perbio), Penicillin/Streptomycin (500 U/mL), glutamine (2 mM), hydrocortisone Upjohn (2.5 mg/L; Serb laboratory), insulin (5 mg/L; Sigma Aldrich). For the differentiation process 2% of DMSO was added to the above-described supplemented Williams medium. Differentiated HepaRG cells (dHepaRG) were infected as previously described (Gripon et al., 2002), with HBV genotype D inoculum prepared either from HepG2.2.15 or HepAD38 cells or with HBV genotype C (GenBank: KP017269.1) prepared from HepG2 cells, which were stably transduced with a linearized pcDNA3-HBV-1.35-genome-unit plasmid and selected on G418 resistance. The multiplicity of infection (MOI; expressed as virus genome equivalent/cell) is indicated in the figure legends, but was of 100 vge/cell if not precised.

An HepaRG-TR-StepTag-HBc cell line was generated by a double transduction with lenviruses carrying the tetracycline repressor (TR) in an expression cassette allowing the resistance to blasticidine and carrying the StrepTag-HBc gene, which encodes the core/capsid protein of HBV with a StrepTag at the N terminus, in an expression cassette allowing the resistance to zeocin. HepaR-TR-StrepTag-HBc cells are cultivated in the same Williams media containing 100 microg/mL of Zeocin and 10 microg/mL of Blasticidin. Tetratcycline is also added to the medium (at 1 microg/mL) to obtain the expression of the transgene, i.e. StepTag-HBc.

StrepTag Purification.

The Strep-Tag® system from IBA, which features a Strep-Tactin affinity column resin, is one of the most widely used affinity chromatography systems for protein purification, detection and immobilization, and offers many advantages, including a high purity of isolated complexes after a physiologic purification process (under buffer condition favorable to protein:protein interaction). The procedure used for the purification is that recommended by manufacturer and can be found on line at: www.ibalifesciences.com/strep-tactin-system-technology.html.

Chemical Reagents.

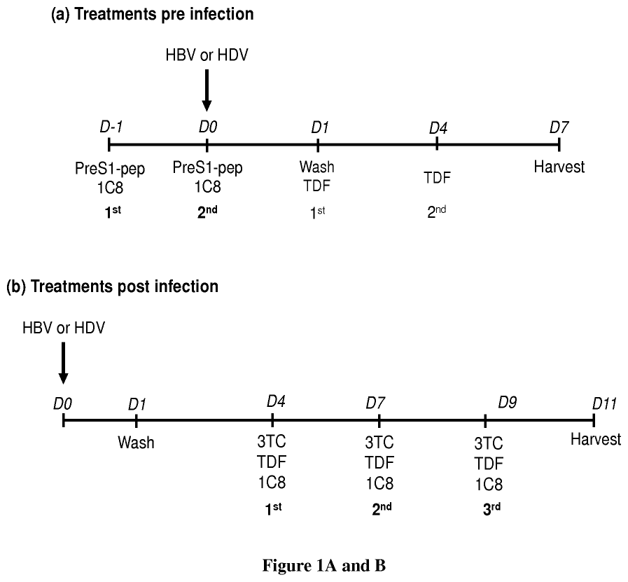

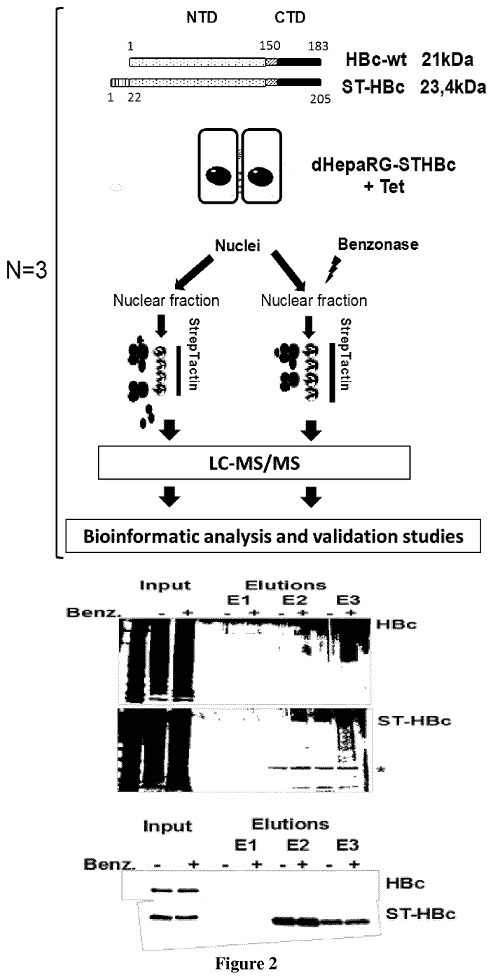

IC8 was provided by Dr Benoit Chabot. The PreS1 peptide (aa sequence 2 to 48 of HBV PreS1, TNLSVPNPLGFFPDHQLDPAFRANSNNPDWDFNPNKDHWPEANKVG (SEQ ID No 1) modified with a myristoyl moiety at N-terminus and resuspended in standard hydrochloride) used for inhibiting HBV entry was synthesized by Genscript (Honk-Hong). Nucleoside analogues, Lamivudine and Tenofovir were kindly provided by Gilead Sciences. The two experimental schemes used to determine the anti-HBV activity of these molecules are presented in FIG. 1.

Cytotoxicity Assay.

Cytotoxicity was measured in HepaRG cells treated with drugs as in experimental schemes presented if FIG. 1 with the “CellTiter-Glo® Luminescent Cell Viability Assay” from Promega, by following manufacturer's instructions.

Analysis of Viral Parameters.

From cell culture supernatants, secreted HBe and HBs antigens were quantified by ELISA, using a chemiluminescence immunoassay kit (Autobio, China) following manufacturer's instructions. Extracellular viral DNA was extracted from cell culture supernatant using MagMAx kit (Thermo scientific) and MagNAPure (Roche) respectively according to manufacturer's protocols and treated either with DNAse-I or RNAse-A. Intracellular total HBV DNA or RNA were extracted from infected cells using respectively Nucleospin 96 Tissue or NucleoSpin 96 RNA kits (Macherey-Nagel). RNAs were transcribed into cDNA using the SuperScript III reverse transcriptase (Invitrogen). Real-time PCR for total intracellular HBV DNA and cDNA from RNA was performed using LightCycler 480 (Roche) and normalized to PRP, a housekeeping gene with the following primers: 5′-GCTGACGCAACCCCCACT-3′ (HBV-sense SEQ ID No 2) and 5′-AGGAGTTCCGCAGTATGG-3′ (HBV-antisense: SEQ ID No 3), 5′-TGCTGGGAAGTGCCATGAG-3′ (PRP-sense: SEQ ID No 4) and 5′-CGGTGCATGTITTCACGATAGTA-3′ (PRPantisens: SEQ ID No 5).

A HBV standard was used for quantification of extracellular viral DNA and RNA. For cccDNA quantification total DNA was digested by T5 exonuclease (New England Biolabs) for 6 hours at 37° C. then submitted to qPCR using Taqman Fast Advanced Master Mix (Life Technologies). CccDNA was normalized to β-Globin quantification. For pgRNA quantification, qPCR was performed using Taqman Fast Advanced Master Mix (Life Technologies) using a home-made probe primer mix. PgRNA levels were normalized to GUSB using a commercial probe primer mix (Life Technologies #Hs99999908_m1).

Statistical Analysis.

Statistical analyses were conducted using non-parametric Mann-Whitney tests using the GraphPad Prism software 6.0. Significance was as follows: *P<0.05, **P<0.01, ***P<0.001, ****P<0.0001.

Results:

SRSF10 is a Major Interactant of the Core/Capsid Protein of HBV

The core/capsid protein of HBV (HBc) is a structural protein, which plays a role in the encapsidation of the HBV pregenomic RNA within the cytoplasm compartment. Following this encapsidation the pgRNA is reverse-transcribed into relaxed circular DNA (rcDNA), which is the genomic form of HBV that is found in virions. Beside this structural function, HBc has potentially other non-structural regulatory functions, in particular in the nucleus of infected cells where it accumulates in highly replicating hepatocytes. To get an insight into the nuclear function of HBc, we performed a mass spectrometry analysis of StepTag-HBc associated protein complexes in the nucleus of tetracycline induced HepaRG-TR-StrepTag-HBc cells. A schematic of the procedure is given on FIG. 2.

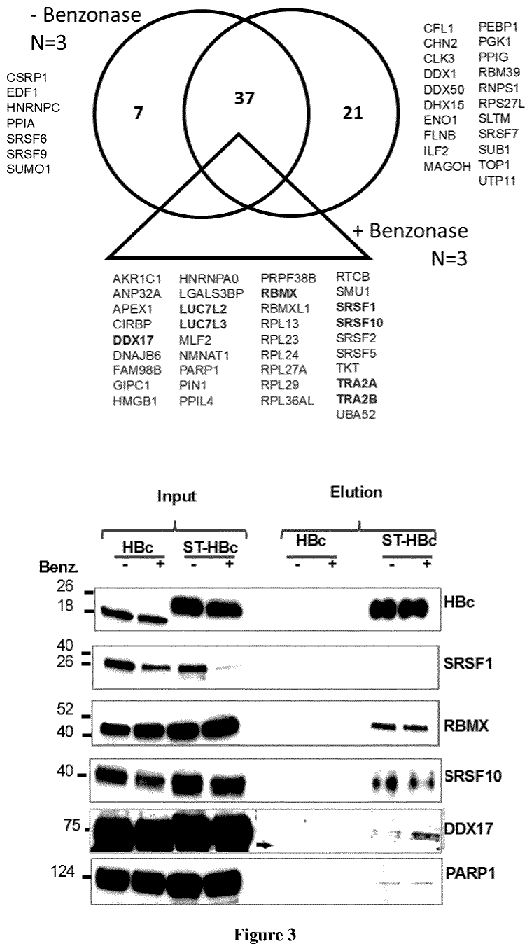

Three independent experiments were performed to increase the statistical power of the analysis. Respectively, 44 and 58 HBc-interacting proteins were found (with a p value <0.005 and an enrichment factor >4) in the benzonase (−) and benzonase (+) conditions. Thirtyseven proteins were common between the two sets of conditions. The list of these proteins is given in FIG. 3.

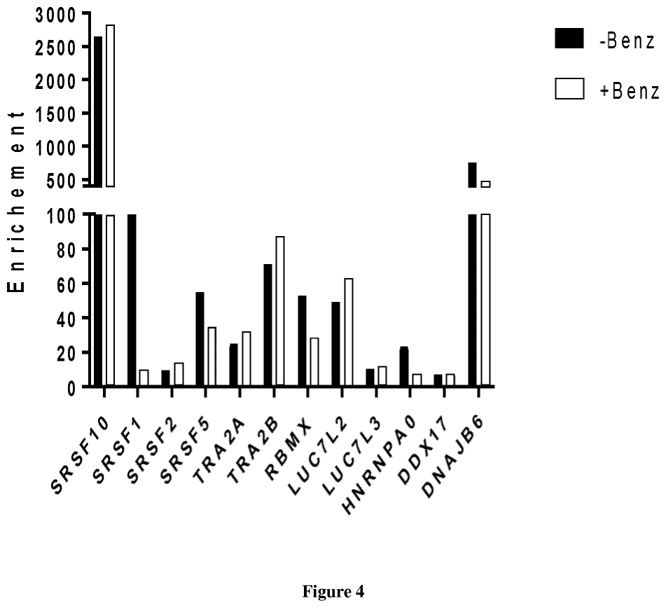

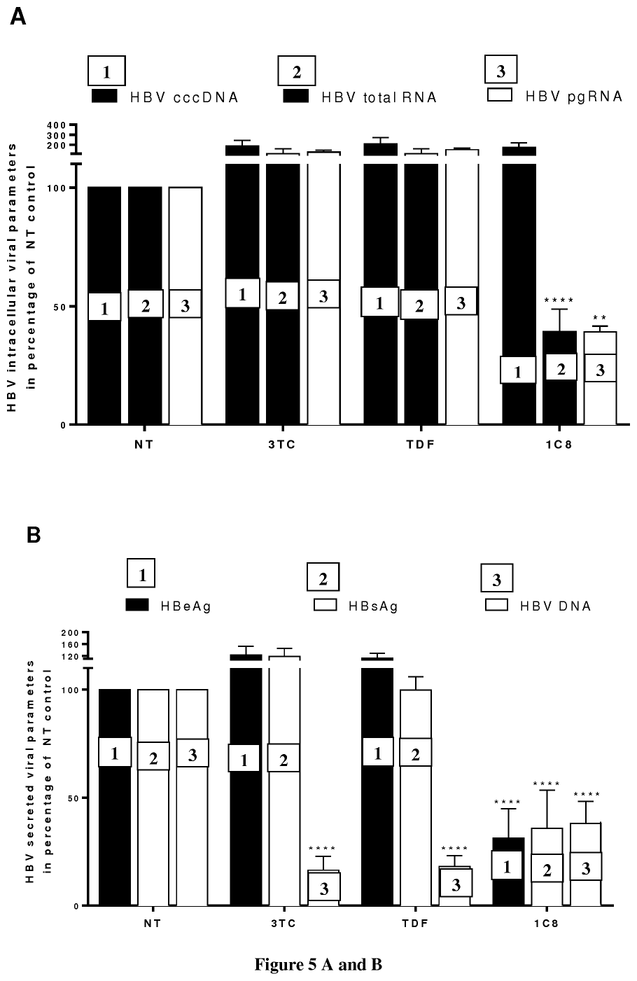

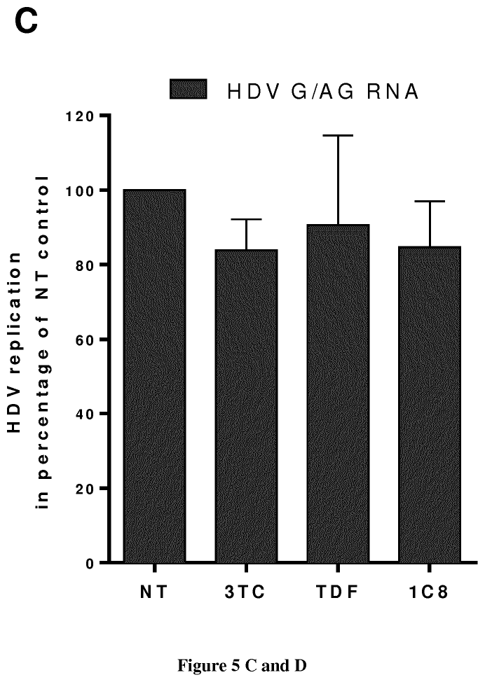

Many HBc interacting proteins were found to be RNA binding proteins involved in RNA metabolism (preRNA maturation, splicing, RNA trafficking . . . ). Among these proteins we got interested in SFSF10, which was one among the most enriched StrepTag-HBc-associated factor, even in the presence of benzonase (FIG. 4). As indicated in the introduction section, this protein was also found to be important for HIV replication. And interestingly, some small molecules with inhibitory properties were previously described, among which 1C8.

Anti-HBV Properties of 1C8 in dHepaRG Cells Replicating HBV

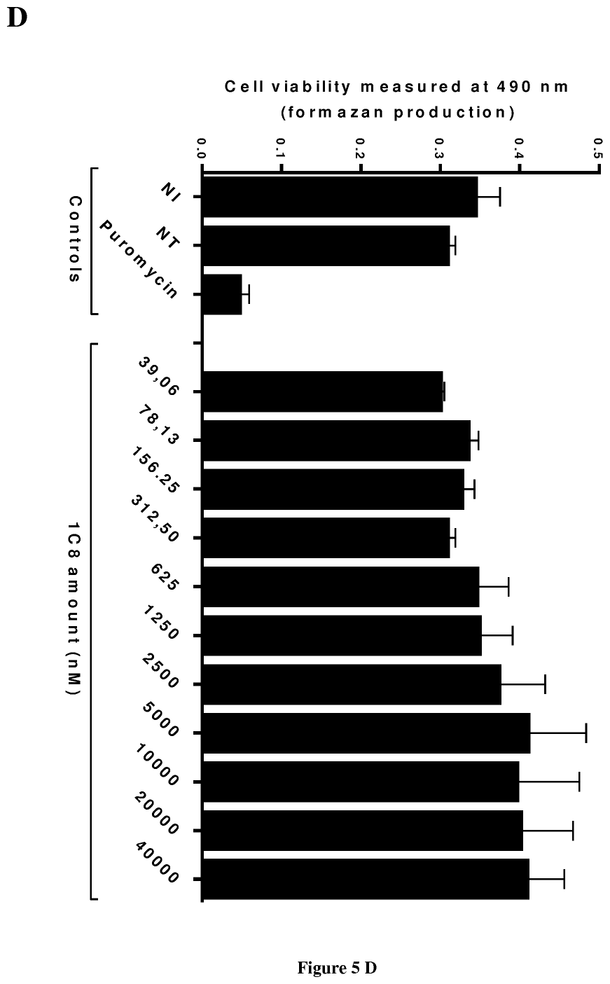

We analysed the potential anti-HBV properties of 1C8 with two different experimental schemes (FIG. 1). In persistently-infected (with HBV genotype D; MOI of 100 vge/cell) dHepaRG treated three times, every 2-3 days, with 10 microM of 1C8 or control molecules, we found that 1C8 was the sole capable to reduce by 60-70% the accumulation of intracellular HBV RNA, when FDA-approved nucleoside analogues (NUC; lamivudine (3TC) and tenofovir (TDF)) were not capable to impact on this viral parameters (FIG. 5A). None of the molecules tested were capable to lower the cccDNA of HBV. 1C8 was also capable to reduce the secretion of viral antigens (HBs and HbeAg) and HBV virions (measured as secreted HBV DNA in FIG. 5B) in cell culture supernatant by 60-70%; this time an expected strong inhibition of HBV virion production was also found with NUCs (FIG. 5B). 1C8 was not capable to inhibit the replication of the hepatitis delta virus in the same cells (FIG. 5C). 1C8 was not toxic for cells up to the 40 microM tested with the CellTiter-Glo® Luminescent Cell Viability Assay (FIG. 5D), thus demonstrating the specificity of the antiviral action against the replication of HBV.

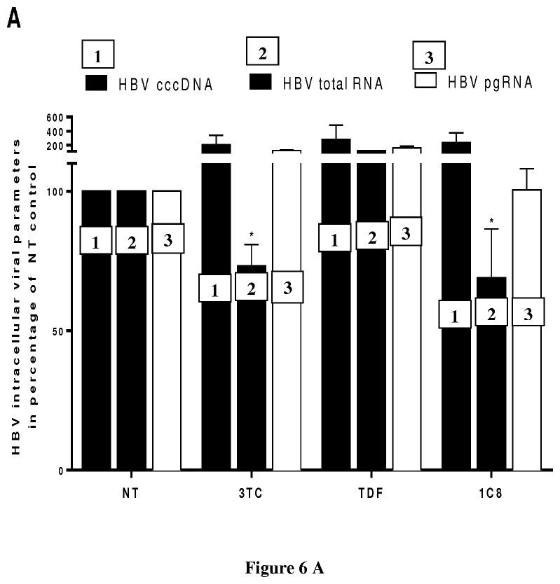

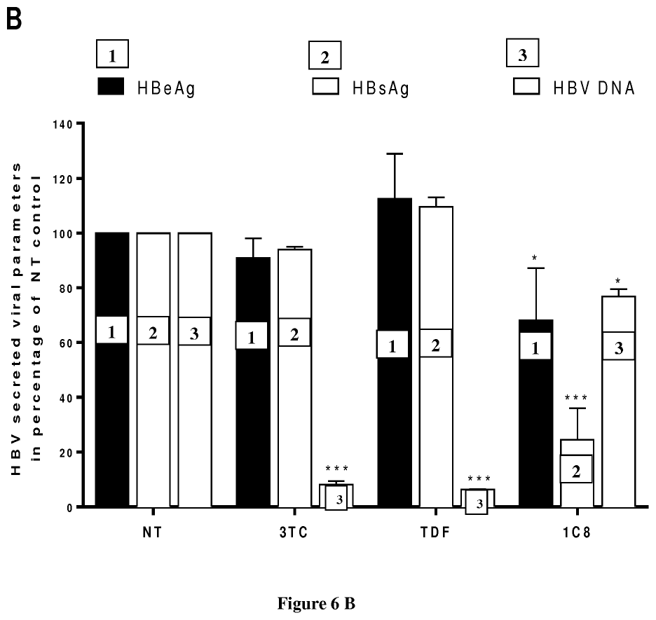

HBV exist as various genotypes. When evaluating host-targeting agents (HTA; i.e. molecules inhibiting a cellular function, which is important for virus replication, and thus lead to antiviral action), like 1C8, it is important to check that the antiviral properties of the HTA is conserved amongst genotypes. We thus evaluated the effect of 1C8 on the replication of a genotype C virus, which is mainly circulating in Asia. We found that 1C8 retained antiviral properties against this strain, although with a slightly reduced efficacy (FIG. 6).

1C8 Impairs Also the Establishment of HBV Infection in Naïve dHepaRG

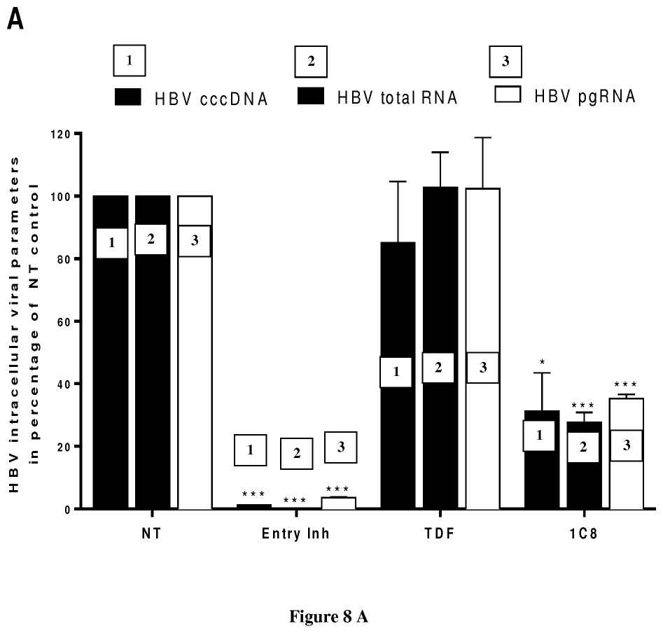

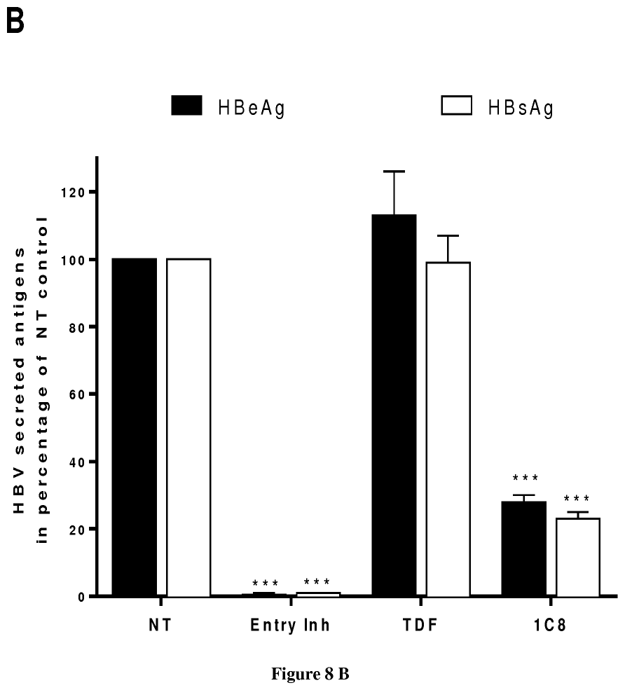

In the second experimental approach we sought to determine whether 1C8 could impair the establishment of HBV infection in dHepaRG cells, like entry inhibitors (Li and Urban, 2016) or core assembly modulators (CAMs) (Berke et al., 2017) do. Entry inhibitors, like PreS1 peptide (e.g. Myrcludex), inhibit the entry of HBV into hepatocytes by preventing the interaction between HBV virion and its cellular receptor, the human sodium taurocholate cotransporting peptide (hNTCP). CAMs, which were primarily developed to inhibit the encapsidation process, were also shown to prevent the establishment of cccDNA by acting at a post-entry level, somehow interfering with the post-entry transport of nucleocapsids toward the nucleus of neo-infected hepatocytes. In contrast, clinically used NUCs do not interfere with the establishment of HBV infection. As the mechanism of anti-HBV inhibition of 1C8 is unknown, we treated cells before and during HBV inoculation with the molecule to determine whether it could prevent the establishment of cccDNA pool and subsequently the synthesis/production of other viral components. We found that 1C8, at 10 microM, was capable to inhibit by 50% the establishment of cccDNA of HBV genotype D (FIG. 8A) and by 70% the establishment of cccDNA of HBV genotype C (FIG. 8A).

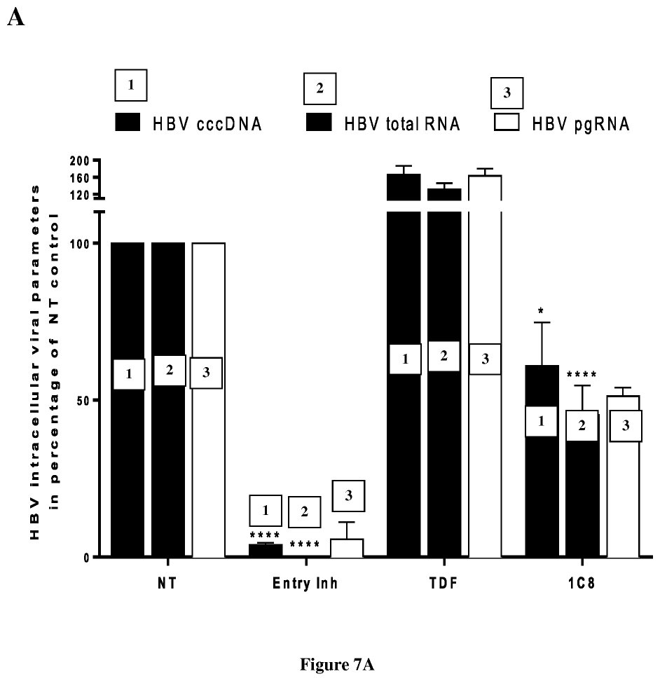

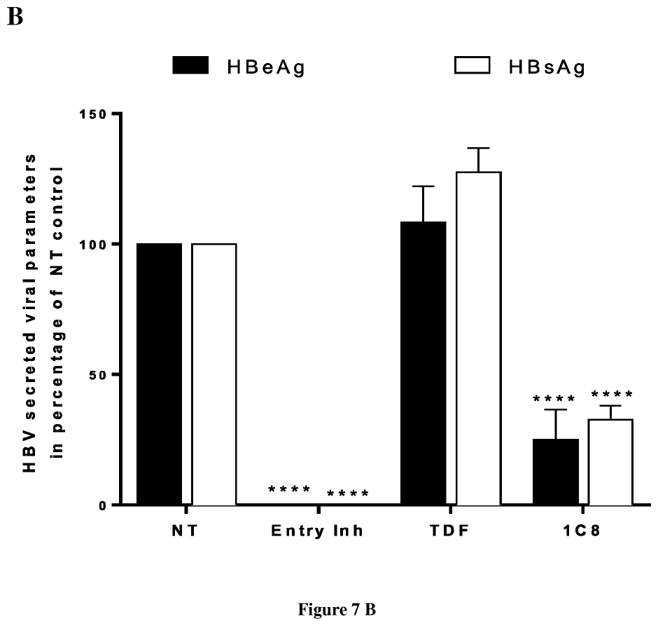

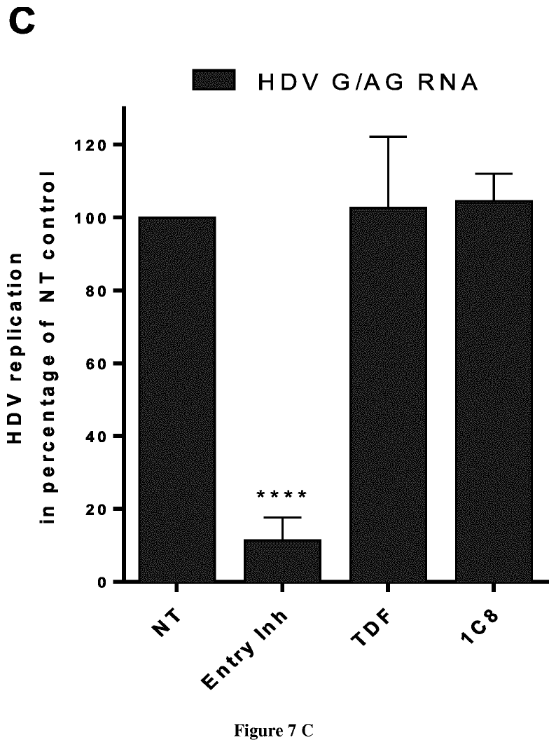

This 1C8-induced inhibition was lower than that obtained with an entry inhibitor, but higher than that obtained with a NUC (tenofovir; TDF). Following the inhibition of cccDNA establishment, other viral markers (i.e. intracellular RNAs, HbeAg, HbsAg) were also reduced in 1C8-treated conditions (FIGS. 7A and B & FIGS. 8A and B). In the mean time and using the same experimental setting (i.e. treatment 24 h before and during inoculation), 1C8 was unable to block the establishment of HDV infections (which was inhibited by the control Entry Inh.), thus suggesting again a specificity of action of 1C8 on HBV (FIG. 7C and FIG. 8C).

### EXAMPLE 2

Material & Methods:

Cultivation of Primary Human Hepatocytes (PHH), and Condition of HBV Infection.

Primary human hepatocytes (PHH) were freshly prepared from human liver resections obtained from the Centre Léon Bérard (Lyon) with French ministerial authorizations as previously described (Leycluse and Alexandre, 2010). They were cultured in Williams medium (Thermofisher) supplemented with 5% of FBS (Perbio), Penicillin/Streptomycin (500 U/mL), glutamine (2 mM), hydrocortisone Upjohn (2.5 mg/L; Serb Laboratory), and insulin (5 mg/L; Sigma Aldrich). PHH were infected as previously described (Gripon et al., 2002), with HBV genotype D inoculum prepared either from HepAD38 cells (GenBank: KP017269.1), at 100 vge/cell. After 16 hours of infection, cells were washed. 6 days post infection, cells were treated with either Tenofovir (TDF), Lamivudine (3TC), or 1C8 at a concentration of 10 μM. Supernatants was harvested after each treatment, and cells were harvested 13 days post infection.

Analysis of Viral Parameters.

Secreted HBe and HBs antigens and intracellular HBV RNA were analyzed as described in EXAMPLE 1.

Statistical Analysis.

Statistical analyses were conducted using non-parametric Mann-Whitney tests using the GraphPad Prism software 6.0. Significance was as follows: *P<0.05, **P<0.01, ***P<0.001, ****P<0.0001.

Results

1C8 Inhibits HBV Replication in Primary Human Hepatocytes

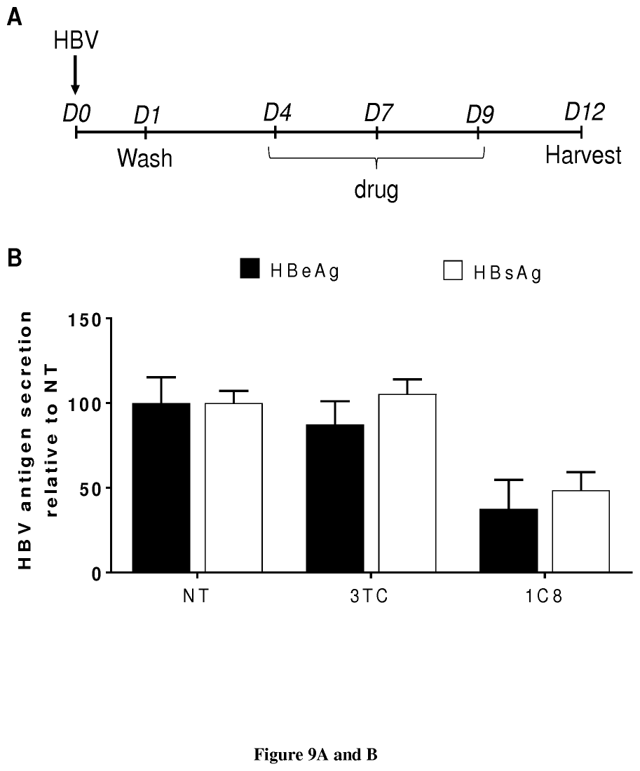

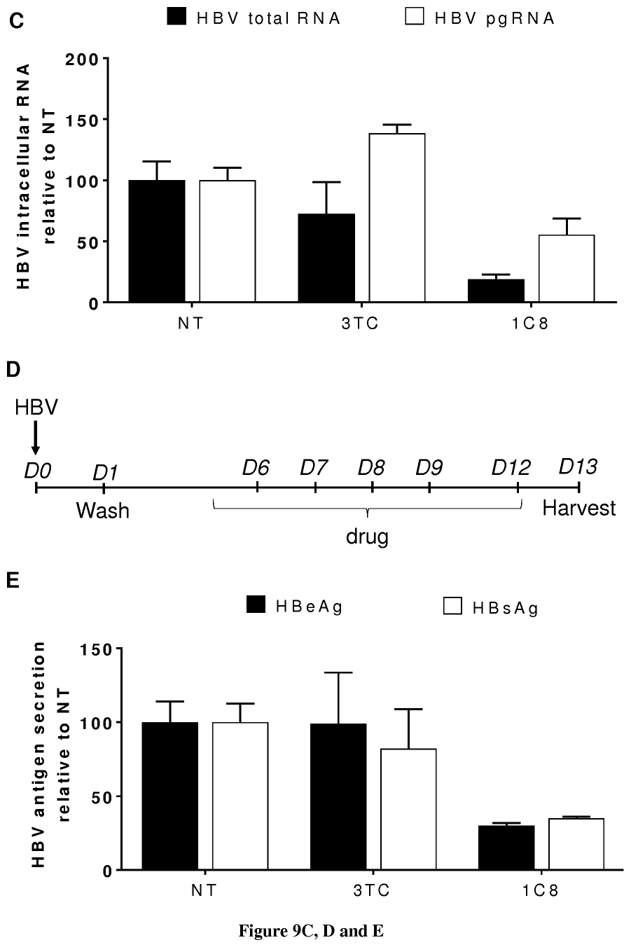

The inhibitory effect of 1C8 was similarly tested on persistently infected PHH. For this, two different batches of PHH, obtained from two different donors, were infected with HBV (with HBV genotype D; MOI of 100 vge/cell) and then treated, with 10 microM of 1C8 or control molecules following two different protocols (FIGS. 9A and D). The analysis of secreted viral antigens, HBe and HBs, indicated that treatment with 1C8 resulted in a robust decrease of viral replications (>60% of decrease) (FIGS. 9B and D). As expected these parameters were not affected by 3TC, a nucleoside analogue that target reverse-transcription of HBV pgRNA. The quantification of intracellular viral RNAs (both total and pgRNA) further indicated that, as previously observed in HepaRG cells (FIG. 6A), addition of 1C8 to HBV infected PHH resulted in a significant reduction of the amount of viral RNAs (FIG. 9C).

Throughout this application, various references describe the state of the art to which this invention pertains. The disclosures of these references are hereby incorporated by reference into the present disclosure.

### REFERENCES

Berke, J. M., Dehertogh, P., Vergauwen, K., Van Damme, E., Mostmans, W., Vandyck, K., and Pauwels, F. (2017). Capsid Assembly Modulators Have a Dual Mechanism of Action in Primary Human Hepatocytes Infected with Hepatitis B Virus. Antimicrobial agents and chemotherapy 61.
Cheung, P. K., Horhant, D., Bandy, L. E., Zamiri, M., Rabea, S. M., Karagiosov, S. K., Matloobi, M., McArthur, S., Harrigan, P. R., Chabot, B., et al. (2016). A Parallel Synthesis Approach to the Identification of Novel Diheteroarylamide-Based Compounds Blocking HIV Replication: Potential Inhibitors of HIV-1 Pre-mRNA Alternative Splicing. J Med Chem 59, 1869-1879.
Gripon, P., Rumin, S., Urban, S., IL Seyec, J., Glaise, D., Cannie, I., Guyomard, C., Lucas, J., Trepo, C., and Guguen-Guillouzo, C. (2002). Infection of a human hepatoma cell line by hepatitis B virus. Proc Natl Acad Sci USA 99, 15655-15660.
Li, W., and Urban, S. (2016). Entry of hepatitis B and hepatitis D virus into hepatocytes: Basic insights and clinical implications. Journal of hepatology 64, S32-40.
Lecluyse, E. L. and Alexandre, E (2010). Isolation and culture of primary hepatocytes from resected human liver tissue. Methods Mol Biol 640, 57-82
Petruzziello, A. (2018). Epidemiology of Hepatitis B Virus (HBV) and Hepatitis C Virus (HCV) Related Hepatocellular Carcinoma. Open Virol J 12, 26-32.
Seeger, C., Zoulim, F., and Mason, W. S. (2015). Hepadnaviruses. In Field's Virology, D. M. Knipe, and P. M. Howley, eds. (Philadelphia: Lippincott Williams & Wilkins), p. 2185.
Shin, C., Feng, Y., and Manley, J. L. (2004). Dephosphorylated SRp38 acts as a splicing repressor in response to heat shock. Nature 427, 553-558.
Shin, C., Kleiman, F. E., and Manley, J. L. (2005). Multiple properties of the splicing repressor SRp38 distinguish it from typical SR proteins. Mol Cell Biol 25, 8334-8343.
Shkreta, L., Blanchette, M., Toutant, J., Wilhelm, E., Bell, B., Story, B. A., Balachandran, A., Cochrane, A., Cheung, P. K., Harrigan, P. R., et al. (2017). Modulation of the splicing regulatory function of SRSF10 by a novel compound that impairs HIV-1 replication. Nucleic Acids Res 45, 4051-4067.
Shkreta, L., Toutant, J., Durand, M., Manley, J. L., and Chabot, B. (2016). SRSF10 Connects DNA Damage to the Alternative Splicing of Transcripts Encoding Apoptosis, Cell-Cycle Control, and DNA Repair Factors. Cell Rep 17, 1990-2003.
Zhou, X., Wu, W., Li, H., Cheng, Y., Wei, N., Zong, J., Feng, X., Xie, Z., Chen, D., Manley, J. L., et al. (2014a). Transcriptome analysis of alternative splicing events regulated by SRSF10 reveals position-dependent splicing modulation. Nucleic Acids Res 42, 4019-4030.
Zhou, X., Wu, W., Wei, N., Cheng, Y., Xie, Z., and Feng, Y. (2014b). Genome-wide analysis of SRSF10-regulated alternative splicing by deep sequencing of chicken transcriptome. Genom Data 2, 20-23.
Zlotnick, A., Venkatakrishnan, B., Tan, Z., Lewellyn, E., Turner, W., and Francis, S. (2015). Core protein: A pleiotropic keystone in the HBV lifecycle. Antiviral Res 121, 82-93.
Zoulim, F., and Durantel, D. (2015). Antiviral therapies and prospects for a cure of chronic hepatitis B. Cold Spring Harb Perspect Med 5.

1. A method of treating Hepatitis B virus (HBV) infection in a subject in need thereof, comprising administering to the subject a therapeutically effective amount of an inhibitor of SRSF10 activity.

2. The method according to claim 1, wherein the HBV infection is a chronic infection.

3. The method according to claim 1, wherein the inhibitor maintains SRSF10 in a dephosphorylated state, and prevents or reduces the splicing activity of SFR10.

4. The method according to claim 1, wherein the inhibitor reduces cccDNA and/or pgRNA in an infected cell.

5. The method according to claim 1, wherein the inhibitor is a small organic molecule selected from the group consisting of: 1C8, E5, D3, C2 and SL309.

6. The method according to claim 5 wherein the small organic molecule is 1C8.

7. A method for screening a plurality of candidate compounds useful for treating Hepatitis B virus (HBV) infection comprising the steps of (a) testing each of the candidate compounds for its ability to inhibit SRSF10 activity and (b) and positively selecting the candidate compounds capable of inhibiting said SRSF10 activity.

8. A pharmaceutical composition for use in a method for treating Hepatitis B virus (HBV) infection in a subject in need thereof, comprising an inhibitor of SRSF10 activity and a pharmaceutically acceptable carrier.

9. (canceled)

10. The pharmaceutical composition of claim 8, wherein the inhibitor of SRSF10 activity is a small organic molecule selected from the group consisting of: 1C8, E5, D3, C2 and SL309.

11. A method of treating Hepatitis B virus (HBV) infection in a subject in need thereof, comprising administering to the subject a therapeutically effective amount of a compound selected from the group consisting of: 1C8 (4-pyridinonebenzisothiazole carboxamide) E5, D3, C2 and SL309.


SEQUENCE LISTING (5 sequences)

SEQ ID NO: 1 - protein, 46 aa, Artificial
  Synthetic PreS1 peptide (aa sequence 2 to 48 of HBV PreS1)
TNLSVPNPLGFFPDHQLDPAFRANSNNPDWDFNPNKDHWPEANKVG

SEQ ID NO: 2 - DNA, 18 bp, Artificial
  Synthetic Primers HBV sense
gctgacgcaacccccact

SEQ ID NO: 3 - DNA, 18 bp, Artificial
  Synthetic Primers HBV antisense
aggagttccgcagtatgg

SEQ ID NO: 4 - DNA, 19 bp, Artificial
  Synthetic Primer PRP sens
tgctgggaagtgccatgag

SEQ ID NO: 5 - DNA, 23 bp, Artificial
  Synthetic Primer PRP-antisens
cggtgcatgttttcacgatagta